(19) United States
(12) Patent Application Publication
(10) Pub. No.: US 2021/0290523 A1
(43) Pub. Date: Sep. 23, 2021

(54) TOPICALLY-APPLIED PROBIOTIC TO TREAT DYSBIOSIS IN THE SKIN MICROBIOME OF PETS AND OTHER ANIMALS

(71) Applicant: Brock LLC, Irvine, CA (US)

(72) Inventors: John Stirling, Irvine, CA (US); Macon Brock, Irvine, CA (US)

(21) Appl. No.: 17/306,442

(22) Filed: May 3, 2021

Related U.S. Application Data
(63) Continuation of application No. 16/039,187, filed on Jul. 18, 2018, now abandoned.
(60) Provisional application No. 62/534,110, filed on Jul. 18, 2017.

(51) Int. Cl. A61K 8/99; A61K 36/064; A61K 35/747; A61Q 19/00; A61Q 5/02

(52) U.S. Cl. CPC A61K 8/99; A61K 36/064; A61Q 17/005; A61Q 19/007; A61Q 5/02; A61K 35/747

(57) ABSTRACT

A topically-applied concoction of prebiotics and probiotics to treat dysbiosis in the skin microbiome of pets and other animals, which includes pet grooming products such as shampoo, conditioner, shampoo & conditioner, leave-in conditioner & detangler, deodorizing spray, or fragrance spray. A related method of treating dysbiosis using a concoction of prebiotics and probiotics is also described. Following up with other topical solutions such as conditioner, deodorizers, sprays that also contain prebiotics and probiotics can be part of the dysbiosis treatment program.

### CROSS REFERENCE TO RELATED APPLICATIONS

This application is a continuation of U.S. application Ser. No. 16/039,187, which claims priority to U.S. Provisional Application No. 62/534,110 filed on Jul. 18, 2017, the disclosure of which, including any materials incorporated by reference therein, are incorporated herein by reference in their entireties.

### BACKGROUND OF THE INVENTION

The present invention generally relates to a method of treating pet skin conditions, in particular, dysbiosis, using a general concoction of topically-applied probiotics and prebiotics included in pet grooming products. Pets can suffer from a variety of conditions caused by disrupted microbial communities (dysbiosis), including excessive shedding, hotspots, dry flaky skin, autoimmune related ailments, and unpleasant odors, for example. Dysbiosis is understood to be a microbial imbalance or maladaptation that is a deviation from a natural state of health of the microbiome. The microbiome is generally understood to be the aggregate of microorganisms that reside on or within the host animal. The present invention addresses the problem of dysbiosis of microbiomes residing on skin, among other things.

The skin of a pet can be home to trillions of pathogenic microorganisms, some good and some bad. Environmental damage and grooming practices and diet can disrupt a pet's skin's natural microbial community, allowing pathogenic microorganisms to flourish, and leaving the animal more prone to bacterial and fungal infection, dry and irritated skin, and autoimmune related ailments, including excessive shedding, dryness, hot spots, infection and excessive odor. Currently, shampoos and conditioners infused with essential oils and extracts are being used to topically help maintain moisture, and sooth irritated skin while the mantle regenerates. However, these oils and conditioners used to allegedly “protect” the skin while the mantle regenerates are poor substitutes and can, in fact, be irritants. Ingestible probiotics are understood to be beneficial to the skin's microbial community but do not supersede the benefits of direct application to the skin as set forth in the present invention.

While probiotics have not reliably been proven to directly treat, prevent or cure disease in animals, per se, dysbiosis of the microbiome commonly found on the surface of the animals' skin has been linked to skin related issues and abnormalities, including, but not limited to, excessive shedding, itching, dryness, hotspots and excessive odor. Prebiotics generally induce growth and/or activity of beneficial microorganisms or microbes and can be derived from a variety of plant sources. The present invention utilizes encapsulated colony forming units of bacterium and yeast and a chemically-preserved delivery system to effectively treat dysbiosis of the microbiome, found on the surface of the host animal s skin. Live colony forming units (CFUs) of beneficial bacterium help to reestablish, support and maintain balance within the natural microbiome of the host organism and restore it to a normal, healthy condition.

Animals are known to also suffer from skin allergies with secondary pyoderma or a bacterial skin infection. In animals with pyoderma, bacteria on the skin triggers the immune system to react by creating an itchy, inflamed, bumpy skid rash.

Currently, no topical probiotic solutions are known to exist for animals or pets to treat dysbiosis of the skin flora and/or pyoderma. The present invention is able to treat these conditions to improve the natural microbiome of the animal's skin to improve the overall well-being of the pet or other animal.

### SUMMARY OF THE INVENTION

In one embodiment, the present invention is directed to a pet shampoo for treating dysbiosis of the microbiome found on the surface of a pet's skin comprising: sulfate-free surfactants; a general concoction of prebiotics and probiotics comprising: Hansenula (Pichia); Kloeckera (Hanseniaspora); Saccharomyces; Lactobacillus; Lactococcus; Leuconostoc Pediococcus; Fig; and Lemon Ferment. The pet shampoo can further comprise water, glycerin, isoeicosane, sorbitan oleate, cellulose gum, chitosan, and citric acid.

In another embodiment, the present invention is directed to a pet grooming product for treating dysbiosis of the microbiome found on the surface of a pet's skin comprising: a prebiotic; and a micro-encapsulated probiotic in dormant colony forming units (CFUs); wherein the pet grooming product being a delivery product being one of a pet shampoo, pet conditioner, pet combination shampoo & conditioner, pet leave-in conditioner and detangler, pet deodorizing spray, fragrance spray. The micro-encapsulated probiotic of the pet grooming product can be Lactobacillus. The pet grooming product can further comprise Hansenula (Pichia), Kloeckera (Hanseniaspora), Lactococcus, Leuconostoc, Pediococcus, Saccharomyces, Fig, and Lemon Ferment.

In a related method, the present invention is directed to a method of treating dysbiosis of the microbiome found on the surface of the host animal's skin, such as excessive shedding, hotspots, dry flaky skin and autoimmune related ailments and unpleasant odors comprises applying a shampoo containing a general concoction of prebiotics and probiotics to a pet's skin; allowing the shampoo containing the probiotic to absorb into the pet's skin for at least one minute; and rinsing the shampoo containing the probiotic from the pet's skin. In the related method, the general concoction comprises: Hansenula (Pichia); Kloeckera (Hanseniaspora); Saccharomyces; Lactobacillus; Lactococcus; Leuconostoc Pediococcus; Fig; and Lemon Ferment.

### DETAILED DESCRIPTION OF THE INVENTION

The present invention provides a topical solution for the treatment of dysbiosis using encapsulated dormant colony forming units (CFUs) of beneficial bacteria in a dormant state. Exemplary delivery products of these topical probiotics include shampoos, conditioners, shampoo & conditioner, leave-in conditioner detangler, deodorizer spray, and fragrance spray/perfume/cologne and other topical solutions.

The topically-applied probiotic in accordance with the present invention comprises of a general concoction, consisting of at least one prebiotic and at least one probiotic, from among the following: Hansenula/Kloeckera/Lactobacillus/Lactococcus/Leuconostoc Pediococcus/Saccharomyces/Fig and Lemon Ferment. As would be understood to those having skill in the art, other compositions of bacterium in support and maintenance of the natural canine, feline, equine, rodent or other animal microbiome could be used and would be within the general scope of the present invention. The specific bacterium utilized may or may not be commonly found in the human or animal biome.

Animals including canine, feline, equine, rodents, primates and general livestock can be treated with the topically applied probiotic in accordance with the present invention. While the probiotic composition does not provide a direct health benefit to the microbiome host, the probiotic composition is understood to provide a health benefit to the microbiome when applied topically to the host animal's skin and/or coat. Any and all benefits are understood to be indirect in nature and the result of a normal and healthy residential bacterial community.

The topically-applied probiotic in accordance with the present invention specifically treats dysbiosis as a result of grooming, anti-bacterial medication or topical treatments, infection, medical conditions, diet, environmental conditions and/or genetic disposition.

The topically applied probiotic in accordance with the present invention may be delivered via a chemically-preserved preparation, usually a common grooming product, such as shampoo, conditioner, detangler or deodorizing spray, but also in any form suitable for application to the skin and/or coat of an animal. Other possible preparations include a hydrating wound spray or balm.

To implement one method in accordance with the present invention, the animal can be washed using the probiotic/prebiotic-infused product, such as shampoo. The washer works the shampoo into a lather and evenly distributes it though the animal's coat and onto the animal's skin. A preferred amount of shampoo is 0.5 fl. oz. per 10 pounds of animal weight. The shampoo is allowed to stay on the animal's skin and absorb into the skin for at least one minute and up to 15 minutes. A preferred amount of time is 2 minutes. The product is then rinsed with water from the animal's skin leaving a protective probiotic layer. A probiotic-infused leave-in conditioner and/or deodorizer can also be applied to introduce additional probiotics.

The introduced probiotics help to support the regeneration of the skin's beneficial pathogenic microorganisms, provide a protective shield against pathogenic microorganisms, and serve to calm the cells that want to react to the presence of pathogenic microorganisms. The probiotics effectively strengthen the skin's natural biological defenses against damage from infection, environmental stresses and styling practices. This can lead to a happier, healthier animal by helping to treat and prevent the five common complaints associated with grooming: hot spots, excessive shedding, dander, odor and infection. Ideally, an animal's skin and coat can be treated bi-weekly, but not less frequently than every four weeks to preserve a clean and natural healthy balance. Leave-in conditioners and deodorizing sprays can be used daily between washings to support a healthy skin mantle.

Probiotics in accordance with the present invention work as both a protective shield and calming agent by displacing the presence of and eliminating the immune system's response to the presence of pathogenic microorganisms. They also help speed up the recovery of and support a healthy skin mantle.

As pet grooming products are already widely used and understood, the present invention provides a convenient and targeted solution to treating these common pet conditions. Further, the probiotic-infused grooming product(s) can be used as a system or as stand-alone additions to an existing grooming regimen. However, the probiotic infused product should be the last product used.

Exemplary probiotics that can be used include, but are not limited to, as generally listed above, include S. thermophiles, Hansenula, Kloeckera, Lactobacillus, Lactococcus, Leuconostoc, Pediococcus, Saccharomyces, Fig, and Lemon Ferment, as found in a milk or sugar kefir.

While the foregoing written description of the invention enables one of ordinary skill in the art to make and use the invention, those of ordinary skill in the art will understand and appreciate the existence of variations, combination, and equivalents of the embodiments, methods, and examples provided herein. The invention should, therefore, not be limited by the embodiments and examples disclosed here, but by all embodiments and methods within the scope and spirit of the invention as claimed.

1. A method of treating dysbiosis of a microbiome found on a surface of a host animal's skin comprising the steps of:
applying a product containing prebiotics and live, micro-encapsulated probiotics in dormant colony forming units to the host animal's skin; and
allowing the product to absorb into the host animal's skin;
wherein the dysbiosis of the microbiome on the surface of the host animal's skin is treated by application of the product containing prebiotics and live, micro-encapsulated probiotics in dormant colony forming units.

2. The method of claim 1, wherein the product includes:
Hansenula;
Kloeckera;
Saccharomyces;
Lactobacillus;
Lactococcus;
Leuconostoc Pediococcus;
Fig; and
Lemon Ferment.

3. The method according to claim 1, wherein the dysbiosis of the microbiome causes ailments such as excessive shedding, matting, hotspots, dry flaky skin and autoimmune-related ailments and unpleasant odors of the host animal's skin.

4. The method of claim 1, wherein the product is one of a pet shampoo, pet conditioner, pet combination shampoo and conditioner, pet leave-in conditioner and detangler, pet deodorizing spray, and fragrance spray.

5. A method of treating dysbiosis of a microbiome found on a surface of a host animal's skin comprising the steps of:
applying a product containing prebiotics and live, micro-encapsulated probiotics in dormant colony forming units to the host animal's skin;
allowing the product to absorb into the host animal's skin; and
rinsing the product from the host animal's skin;
wherein the dysbiosis of the microbiome on the surface of the host animal's skin is treated by application of the product containing prebiotics and live, micro-encapsulated probiotics in dormant colony forming units.

6. The method of claim 5, wherein the product includes:
Hansenula;
Kloeckera;
Saccharomyces;
Lactobacillus;
Lactococcus;
Leuconostoc Pediococcus;
Fig; and
Lemon Ferment.

7. The method according to claim 5, wherein the dysbiosis of the microbiome causes ailments such as excessive shedding, matting, hotspots, dry flaky skin and autoimmune related ailments and unpleasant odors of the host animal's skin.

8. The method of claim 5, wherein the product is one of a pet shampoo, pet conditioner, and pet combination shampoo & conditioner.
